(19) United States
(12) Patent Application Publication
(10) Pub. No.: US 2008/0223361 A1
(43) Pub. Date: Sep. 18, 2008

(54) Respiratory medicine delivery system

(76) Inventor: Peter Nieuwstad, Seattle, WA (US)

Correspondence Address: LITMAN LAW OFFICES, LTD., P.O. BOX 15035, CRYSTAL CITY STATION, ARLINGTON, VA, 22215, US

(21) Appl. No.: 12/068,065

(22) Filed: Feb. 1, 2008

Related U.S. Application Data
(60) Provisional application No. 60/906,852, filed on Mar. 14, 2007.

(51) Int. Cl. A61M 11/00

(52) U.S. Cl. 128/200.14

(57) ABSTRACT

The respiratory medicine delivery system is a handheld breathing device for a user having respiratory problems. The device comprises a hollow tubular manifold. A front tubular section extends from a front opening of the hollow tubular manifold. A mouthpiece extends from a rear opening of the hollow tubular manifold. The front tubular section has an MDI port. A seal cap that can seal off the MDI port is provided. A PEEP valve is mounted in the front tubular section to create a sealed backpressure in the user's airways by creating resistance to exhalation. The hollow tubular manifold has a manifold inlet. A threaded nebulizer port having a recessed one-way valve can be formed at the inlet of the manifold. The nebulizer port is provided for attachment to a nebulizer to facilitate buildup of positive pressure and delivery of medication to a user's lungs. A nebulizer seal cap is also provided.

### CROSS-REFERENCE TO RELATED APPLICATION

This application claims the benefit of U.S. Provisional Patent Application Ser. No. 60/906,852, filed Mar. 14, 2007.

### BACKGROUND OF THE INVENTION

1. Field of the Invention

The present invention relates to devices for delivering medication to the airways of a patient, and more particularly to a respiratory medicine delivery device having a positive end-expiratory (PEEP) valve and ports for connection to a nebulizer or a metered dose inhaler (MDI).

2. Description of the Related Art

People who have been diagnosed with chronic obstructive pulmonary disease (COPD) often must inhale medicine to relieve symptoms of the disease. For example, Metered Dose inhalers (MDI) are used to assist the patient, relieving symptoms of coughing, sputum expectoration, shortness of breath (SOB), and the like. MDIs are generally lightweight, compact dosing units that can deliver an aerosol of medication to the COPD sufferer when the MDI mouthpiece is puffed on. When a COPD sufferer cannot achieve relief from the symptoms by using an MDI, the next step is to either go to the hospital or home for treatment with a nebulizer. While a nebulizer is generally bulky, it delivers liquefied medicine to the lungs for quick relief of the COPD symptoms.

In the event that the COPD sufferer does not have his/her medicine handy on the trip to obtain treatment, or the medicine has run out, the patient generally follows a routine of exhaling with pursed lips so that positive pressure is applied to the lung air passageways in order to keep them open to avoid excess CO₂ buildup in the blood stream. Alternatively, the COPD sufferer may have in possession a positive end expiratory pressure (PEEP) valve to achieve the appropriate positive pressure on the lungs when exhaling.

Thus, a COPD sufferer may require up to least three devices for control of the COPD symptoms. It would be highly beneficial to combine the functionality of an MDI, a nebulizer, and a PEEP valve in one easily configurable device.

Thus, a respiratory medicine delivery system solving the aforementioned problems is desired.

### SUMMARY OF THE INVENTION

The respiratory medicine delivery system is a handheld breathing device for a user having respiratory problems. The device comprises a hollow tubular manifold. A front tubular section extends from a front opening of the hollow tubular manifold. The front tubular section defines an air intake port. A mouthpiece extends from a rear opening of the hollow tubular manifold.

The front tubular section has an MDI port. A seal cap that can seal off the MDI port is provided. A PEEP valve is mounted in the front tubular section to create a sealed backpressure in the user's airways by creating resistance to exhalation. The hollow tubular manifold has a manifold inlet.

A threaded nebulizer port can be formed at the inlet of the manifold. The nebulizer port is provided for attachment to a nebulizer to facilitate buildup of positive pressure and delivery of medication to a user's lungs. A nebulizer seal cap is also provided.

These and other features of the present invention will become readily apparent upon further review of the following specification and drawings.

### BRIEF DESCRIPTION OF THE DRAWINGS

FIG. 1 is a front perspective view of the respiratory medicine delivery system according to the present invention.

FIG. 2 is a rear perspective view of the respiratory medicine delivery system according to the present invention.

FIG. 3 is an exploded perspective view of the respiratory medicine delivery system according to the present invention.

FIG. 4 is a rear elevational view of the respiratory medicine delivery system according to the present invention.

FIG. 5 is a front elevational view of the respiratory medicine delivery system according to the present invention.

FIG. 6 is a perspective view of the respiratory medicine delivery system with integrated PEEP valve, according to the present invention.

Similar reference characters denote corresponding features consistently throughout the attached drawings.

### DETAILED DESCRIPTION OF THE PREFERRED EMBODIMENT

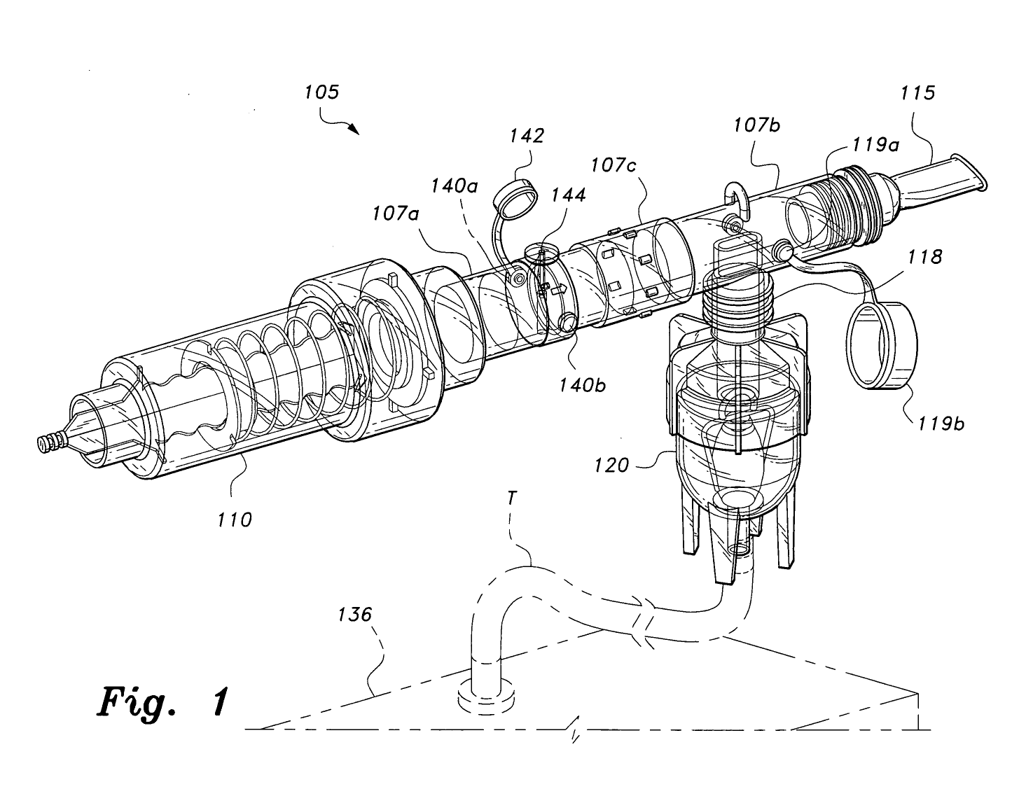

As shown in FIG. 1, the present invention is a respiratory medicine delivery system that includes a handheld breathing device 105 for use by a person having respiratory problems. The device comprises a hollow tubular manifold 107b. A front tubular section 107a extends from a front opening of the hollow tubular manifold 107b. A spacer 107c can be used to facilitate the extension of tubular section 107a from the hollow tubular manifold 107b. A mouthpiece 115 extends from a rear opening of the hollow tubular manifold 107b.

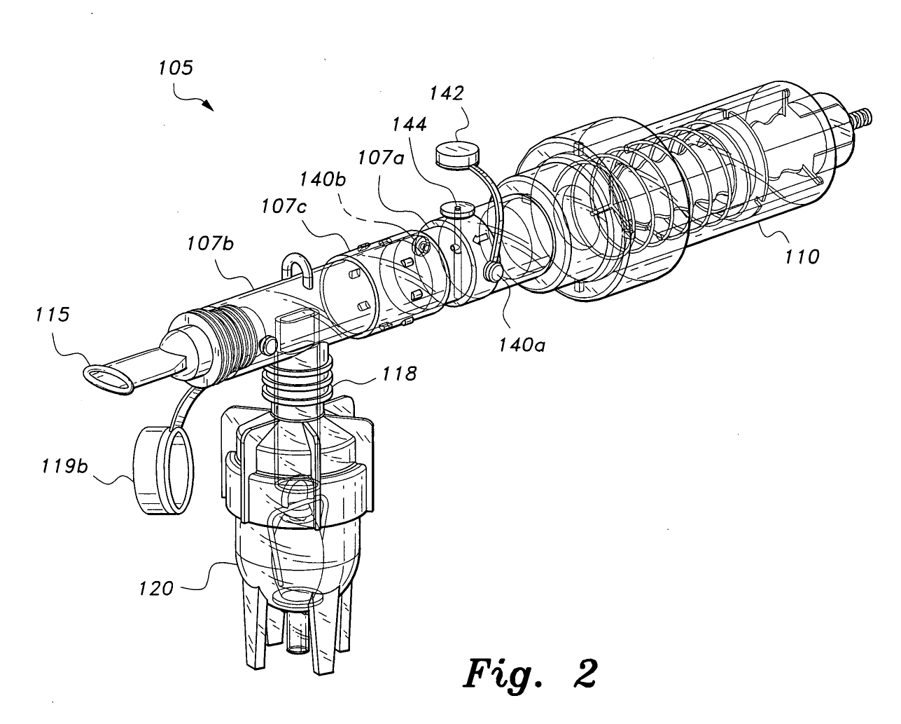

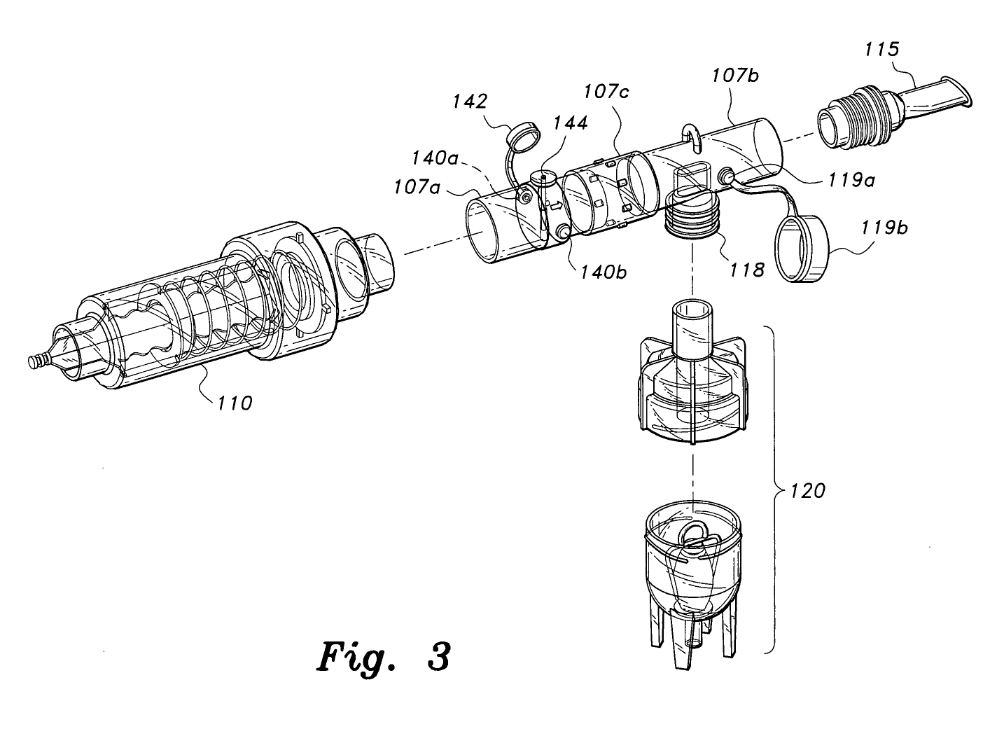

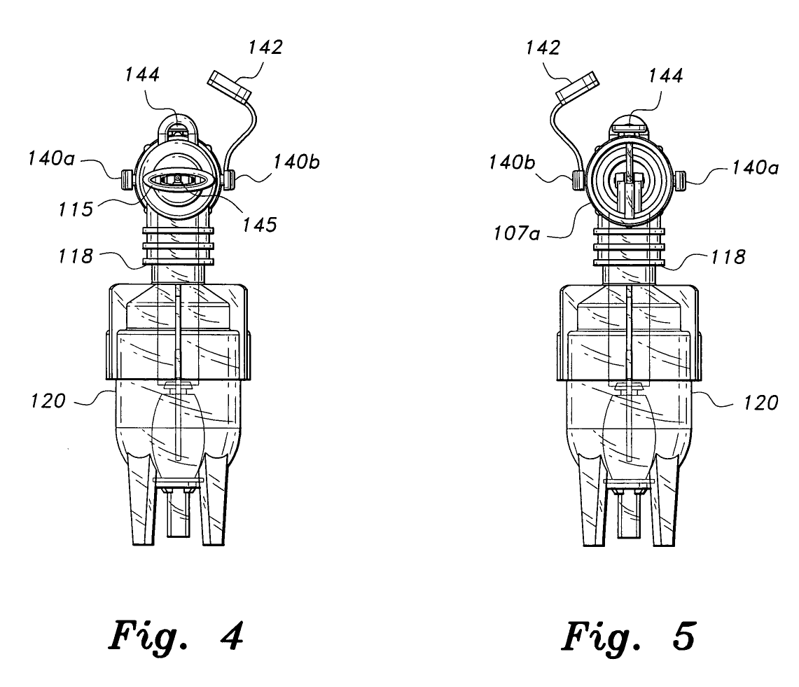

The front tubular section 107a has an MDI port 144. As shown in FIGS. 1-3, the MDI port 144 comprises a top opening that can accommodate an MDI inhaler filled with medicine. The MDI port 144 has a stem that extends into the body of tubular section 107a. As shown in FIGS. 3-4, the stem has an opening 145 disposed within the body of tubular section 107a and directed towards the mouthpiece 115 so that an inhalant mist of medicine can travel towards the user as he/she inhales through the mouthpiece 115. As shown in FIGS. 1-5, a seal cap 142 that can seal off MDI port 144 is also provided. The seal cap can be retained by one of two protruding knobs 140a and 140b disposed on the front tubular section 107a.

A PEEP valve 110 can be mounted in the front tubular section 107a to create a sealed backpressure in the user's airways by creating resistance to exhalation. The PEEP valve 110 may be a removably attached unit, or may be integrated within the front tubular section 107a. The PEEP valve 110 may preferably include an air intake valve integrated within the unit. Alternatively, a one-way expiratory PEEP valve can be used, in which case an additional inspiratory one-way air entrainment valve can be disposed elsewhere on the medicine delivery device 105.

Hollow tubular manifold 107b has a manifold inlet. As most clearly shown in FIG. 3, a threaded nebulizer port 118 can be formed at the inlet of the manifold 107b. The nebulizer port 118 can be sealed off by nebulizer seal cap 119b when not in use. Nebulizer seal cap 119b is retained on the device 105 by nebulizer seal cap retaining knob 119a. The nebulizer port 118 provides a removable attachment for a nebulizer, such as nebulizer 120. As shown in FIG. 1, when the nebulizer 120 is attached to the delivery system 105, a tube T can connect to a liquid medicine reservoir 136 to facilitate buildup of positive pressure and delivery of liquid medication to a user's lungs.

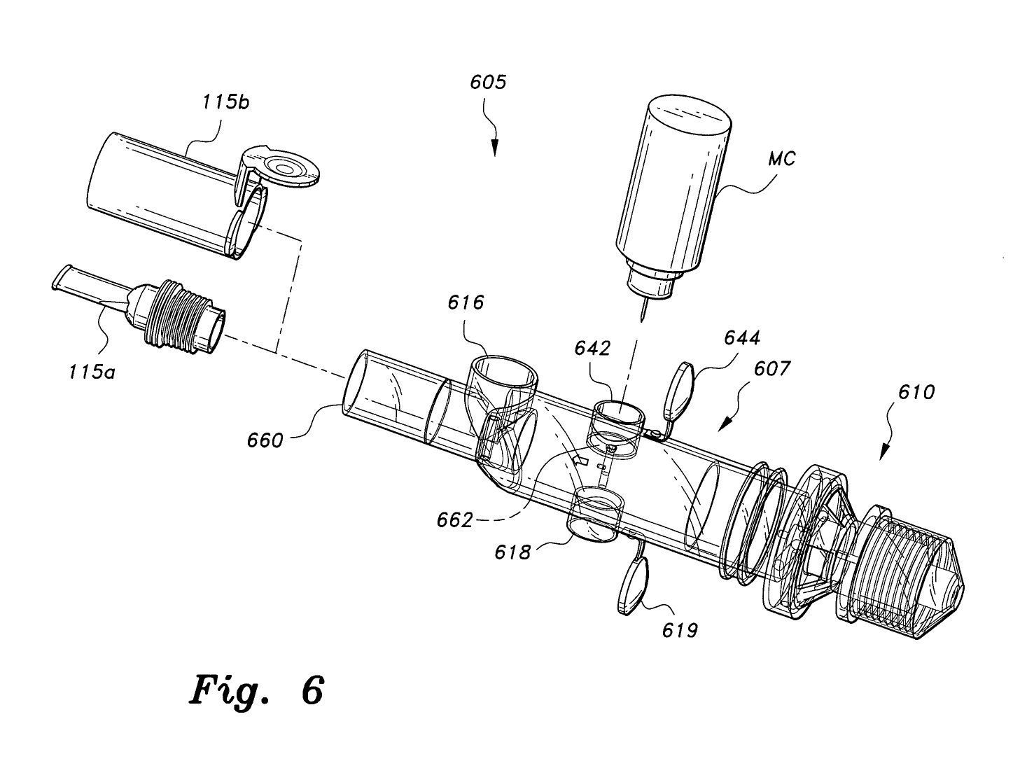

As discussed supra, and most clearly shown in FIG. 6, an alternative embodiment handheld breathing device 605 can have an integrated tubular manifold 607 and PEEP device 610. As in device 105, the unit has an MDI port 642, a cap 644 for the MDI port, a nebulizer port 618, a nebulizer port cap 619. Moreover, the MDI port 642 may have a shelf 662 disposed across the opening in the manifold 607. Preferably, there may by approximately ⅛″ space between the neck of an MDI inhaler (medication container) MC and the internal shelf 662 so that when depressed and activated, the neck of the medication container MC can seal against the shelf 662 to seal the port for exhalation. Additionally, the device 605 has an air intake port 616 disposed on the tube 607 between MDI port 642 and mouthpiece end, i.e., free end 660, of the tube 607. Preferably, air intake port 616 is an air entrainment inspiratory one-way valve. Mouthpiece 115a or end cap 115b can be fitted at the free end 660 of the tube 607.

Thus, the system described in the present invention is capable of delivering an MDI dose, creating positive expiratory pressure in the user's lungs, delivering a nebulizer dose, and may be used as a standalone breathing device during a bronchospasdic attack.

It is to be understood that the present invention is not limited to the embodiment described above, but encompasses any and all embodiments within the scope of the following claims.

I claim:

1. A respiratory medicine delivery system, comprising:
a hollow tubular manifold having a front opening and a rear opening;
a front tubular section extending from the front opening of the manifold, the front tubular section defining an air intake port;
a mouthpiece extending from the rear opening of the manifold;
a Metered Dose Inhaler (MDI) intake port opening into the front tubular section;
an MDI seal cap removably attached to the MDI port for covering the MDI intake port when the MDI intake port is not in use;
a Positive End-Expiratory (PEEP) valve disposed in the front tubular section, the PEEP valve regulating resistance to air exhaled by a patient;
a nebulizer intake port connected to the manifold inlet for attaching a nebulizer; and
a nebulizer intake seal cap removably attached to the nebulizer port for covering the nebulizer intake port when the nebulizer intake port is not in use.

2. A respiratory medicine delivery system, comprising:
a hollow tubular manifold having a plurality of manifold openings;
a Positive End-Expiratory (PEEP) valve, the PEEP valve being unitarily connected to an axial end of the hollow tubular manifold, the PEEP valve regulating resistance to air exhaled by a patient;
a Metered Dose Inhaler (MDI) intake port formed by a first one of the plurality of openings defined by the manifold, the MDI intake port accepting a medication container;
an air intake port formed by a second one of the plurality of manifold openings;
an air entrainment inspiratory one way valve disposed inside the air intake port; and
a mouthpiece, the mouthpiece being attached to an axial end of the tubular manifold.

3. The respiratory medicine delivery system according to claim 2, further comprising a shelf disposed in the MDI intake port, the shelf forming an exhalation seal when the shelf is engaged with the medication container.

4. The respiratory medicine delivery system according to claim 2, wherein the MDI intake port includes a stem extending into the tubular manifold, the stem having an opening directed towards the mouthpiece, the stem opening being adapted for directing an inhalant mist of medicine towards a patient as the patient inhales through the mouthpiece.

5. The respiratory medicine delivery system according to claim 2, further comprising a cap disposed on the hollow tubular manifold, the cap sealing the MDI intake port when a user places the cap over the MDI intake port.

6. The respiratory medicine delivery system according to claim 2, wherein a third one of the plurality of manifold openings forms a nebulizer port, the system further comprising a nebulizer port cap disposed on the hollow tubular manifold, the nebulizer port cap sealing the nebulizer port when a user places the nebulizer port cap over the nebulizer port.

7. The respiratory medicine delivery system according to claim 2, wherein the mouthpiece is removably attached to the tubular manifold.

8. The respiratory medicine delivery system according to claim 7, further comprising an end cap attachable to the manifold in place of the removable mouthpiece.
